(19) United States
(12) Patent Application Publication
(10) Pub. No.: US 2006/0190042 A1
(43) Pub. Date: Aug. 24, 2006

(54) Tissue repair assembly

(75) Inventors: Kevin T. Stone, Winona Lake, IN (US); Ryan A. Kaiser, Leesburg, IN (US); Zachary M. Hoffman, Warsaw, IN (US)

Correspondence Address: HARNESS, DICKEY & PIERCE, P.L.C., P.O. BOX 828, BLOOMFIELD HILLS, MI, 48303, US

(73) Assignee: Arthrotek, Inc., Warsaw, IN (US)

(21) Appl. No.: 11/347,662

(22) Filed: Feb. 3, 2006

Related U.S. Application Data
(63) Continuation-in-part of application No. 10/983,236, filed on Nov. 5, 2004, now abandoned.

(51) Int. Cl. A61B 17/04

(52) U.S. Cl. 606/232

(57) ABSTRACT

A tissue repair assembly for repairing tissue. The tissue repair assembly includes a rotatable anchor for insertion through a tissue tear on a surface of the tissue, a threaded fastener for insertion into the tissue, the threaded fastener having a longitudinal bore and a plurality of apertures communicating with the bore, and a flexible member coupled to the rotatable anchor and passing through the bore such that tensioning the flexible member compresses the tear.

### CROSS-REFERENCE TO RELATED APPLICATIONS

This application is a continuation-in-part of U.S. patent application Ser. No. 10/983,236 filed on Nov. 5, 2004. The disclosure of the above application is incorporated herein by reference.

### INTRODUCTION

Tears caused by trauma or disease in soft tissue, such as cartilage, ligament, or muscle, can be repaired by suturing. Various devices have been developed for facilitating suturing and are effective for their intended purposes. Nevertheless, tissue repair assemblies for facilitating suturing are still desirable.

### SUMMARY

The present teachings provide a tissue repair assembly. The tissue repair assembly includes first and second rotatable anchors for insertion through a tissue tear on a surface of the tissue, and a flexible member connecting the first and second anchors. The flexible member defines a pre-assembled self-locking slip knot having a free end, such that tensioning the free end compresses the tear and locks the slip knot.

The present teachings also provide a method for repairing a tear in tissue. The method includes providing an inserter having a shaft loaded with first and second rotatable anchors, wherein the anchors are connected with a flexible member, and the flexible member defines a self-locking slip knot and has a free end. The method also includes deploying the first anchor through a first surface of the tissue across the tear onto a second surface of the tissue, deploying the second anchor through the first surface of the tissue across the tear onto the second surface of the tissue at a distance apart from the first anchor, and tensioning the free end of the flexible member to reduce the tear.

The present teachings also provide a tissue repair assembly that includes an inserter for insertion through a tissue tear on a surface of the tissue, a shaft coupled to the inserter, first and second anchors loaded into the shaft, wherein each anchor includes slanted ends and a curved projection for rotating the anchor upon deployment from the inserter, and a flexible member connecting the first and second anchors. The flexible member defines a pre-assembled self-locking slip knot having a free end, such that tensioning the free end compresses the tear and locks the slip knot.

The present teachings further provide a tissue repair assembly that includes an inserter for insertion through a tissue tear on a surface of the tissue, a shaft coupled to the inserter, at least one anchor having an end loop coupled thereto, wherein the anchor is loaded to the inserter for deployment therefrom, and a flexible member defining a pre-assembled self-locking slip knot. The flexible member has a free end passing through the end loop, such that tensioning the free end compresses the tear and locks the slip knot.

The present teachings also provide a method for repairing a tear in tissue including providing an inserter having a shaft loaded with a rotatable anchor having an end loop coupled thereto, and a flexible member passing through the end loop, wherein the flexible member defines a self-locking slip knot and has a free end. The method also includes deploying the anchor at a first location through a first surface of the tissue across the tear to a second surface of the tissue, inserting the inserter through the first surface of the tissue across the tear to the second surface of the tissue at a second location a distance apart from the anchor, capturing the free end of the flexible member, withdrawing the free end of the flexible member through the second location, passing the free end of the flexible member though the slip knot, and tensioning the free end of the flexible member to reduce the tear.

The present teachings further provide a method for repairing a tear in tissue using a plurality of anchors. The method includes providing a flexible member having a pre-tied slip knot attached to a suture passer, wherein the flexible member has a free end, passing the free end of the flexible member through a first anchor from the plurality of anchors, inserting the first anchor through a first surface of the tear onto a second surface of the tear at a first location, passing the free end of the flexible member through a second anchor from the plurality of anchors, inserting the second anchor through the first surface of the tear on the second surface of the tear at a second location, repeating inserting and passing until all the anchors from the plurality of anchors are inserted on the second surface of the tear, capturing the free end of the flexible member, withdrawing the free end of the flexible member through the tear, passing the free end of the flexible member though the slip knot, and tensioning the free end of the flexible member to reduce the tear.

The present teachings also provide a tissue repair assembly including a rotatable anchor for insertion through a tissue tear on a surface of the tissue, a threaded fastener for insertion into the tissue, the threaded fastener having a longitudinal bore and a plurality of apertures communicating with the bore, and a flexible member coupled to the rotatable anchor and passing through the bore such that tensioning the flexible member compresses the tear.

The present teachings also provide a method for repairing a tear in tissue using a plurality of anchors. The method includes providing a suture passer with a flexible member having a pre-tied slip knot coupled to the suture passer, wherein the flexible member has a free end, inserting each of the plurality of anchors through a first surface of the tear onto a second surface of the tear such that the flexible member passes through the tear before passing through a subsequent anchor, passing the free end of the flexible member though the slip knot after inserting each of the plurality of anchors, and tensioning the free end of the flexible member to reduce the tear.

The present teachings provide a method for repairing a tear in tissue including providing an inserter having a shaft loaded with a rotatable anchor and a threaded fastener. The rotatable anchor and the threaded fastener are connected with a flexible member passing through a bore of the fastener. The flexible member defines a self-locking slip knot outside the fastener and has a free end. The method further includes deploying the rotatable anchor through a first surface of the tissue across the tear onto a second surface of the tissue, deploying the threaded fastener through the first surface of the tissue across the tear into the tissue, and tensioning the free end of the flexible member to reduce the tear.

The present teaching provide a method for repairing a tear in tissue including providing a plurality of rotatable anchors and a threaded fastener connected with a flexible member. The flexible member passes through a bore of the threaded fastener and defines loop terminating at a self-locking slip knot. The method includes inserting each rotatable anchor through a first surface of the tear onto a second surface of the tear such that the flexible member passes through the tear between successive anchors, inserting the threaded fastener through the first surface of the tear into the tissue, tensioning the free end of the flexible member to reduce the tear and lock the slip-knot against the fastener, and allowing biological fluid flow through the bore of the threaded fastener and apertures thereon.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an environmental view of a repair assembly according to the present teachings;

FIG. 2 is a side view of an anchor of the repair assembly of FIG. 1;

FIG. 3 is plan view of the anchor of FIG. 2;

FIG. 4 is an environmental view showing an insertion direction for the anchor of FIG. 2;

FIG. 5 is an environmental view showing an implantation direction for the anchor of FIG. 2;

FIGS. 6-9 illustrate forming a first end loop coupled to a first anchor of a repair assembly according to the present teachings;

FIGS. 10-17 illustrate forming a slip knot in a flexible member connecting first and second anchors of a repair assembly according to the present teachings;

FIG. 17A illustrates a repair assembly according to the present teachings;

FIG. 17B illustrates a repair assembly according to the present teachings;

FIG. 18 is a perspective view of an inserter of a repair assembly according to the present teachings;

FIG. 19 is a perspective view of a shaft for the inserter of FIG. 18, shown with first and second anchors loaded thereon;

FIG. 20 is a plan view of an inserter of a repair assembly according to the present teachings;

FIG. 21 is a sectional view of the inserter of FIG. 20 taken along the line 21-21;

FIG. 22 is an enlarged view of Detail B of FIG. 21;

FIG. 23 is a sectional view of a portion of a shaft of a repair assembly according to the present teachings, shown with first and second anchors loaded thereon;

FIG. 24 is an environmental view of a calibrated probe for determining an insertion depth;

FIG. 25 is an environmental view showing the repair assembly according to the present teachings before implantation of a first anchor;

FIG. 26 is an environmental view of the repair assembly of FIG. 25 shown after implantation of the first anchor and before implantation of the second anchor;

FIG. 27 is an environmental view of the repair assembly of FIG. 25 shown after implantation of the first anchor and second anchors and before tensioning the flexible member of the repair assembly;

FIG. 28 is an environmental view of the repair assembly of FIG. 25, shown after implantation of the first anchor and second anchors and after tensioning the flexible member of the repair assembly;

FIG. 29 illustrates a repair assembly according to the present teachings;

FIG. 30 is a plan view of a portion of an inserter for a repair assembly according to the present teachings;

FIG. 31 is a side view of a capturing probe for the inserter of FIG. 30;

FIG. 32 is a side view of the capturing probe of FIG. 31 loaded on the inserter of FIG. 30;

FIG. 33 is a plan view of the capturing probe of FIG. 31 loaded on the inserter of FIG. 30;

FIG. 34 is a detail illustration an anchor with an end loop for a repair assembly according to the present teachings;

FIG. 35 illustrates a slip knot in the process of being tied on a suture passer for a repair assembly according to the present teachings;

FIG. 36 illustrates the slip knot of FIG. 36 after tensioning for tying;

FIG. 37 illustrates a method of reducing a tear using one anchor according to the present teachings;

FIG. 38 illustrates a method of reducing a tear using a plurality of anchors according to the present teachings;

FIG. 39 is an environmental view of a repair assembly according to the present teachings;

FIG. 40 is an environmental view of a repair assembly according to the present teachings; and

FIG. 41 is an environmental view of a repair assembly according to the present teachings.

### DESCRIPTION OF VARIOUS EMBODIMENTS

The following description is merely exemplary in nature and is in no way intended to limit the invention, its application, or uses. For example, although the present teachings are illustrated in an application for meniscus repair in knee surgery, the present teachings can also be used for repairing any soft tissue, such as muscle, ligament or tendon in an arthroscopic or other open procedure.

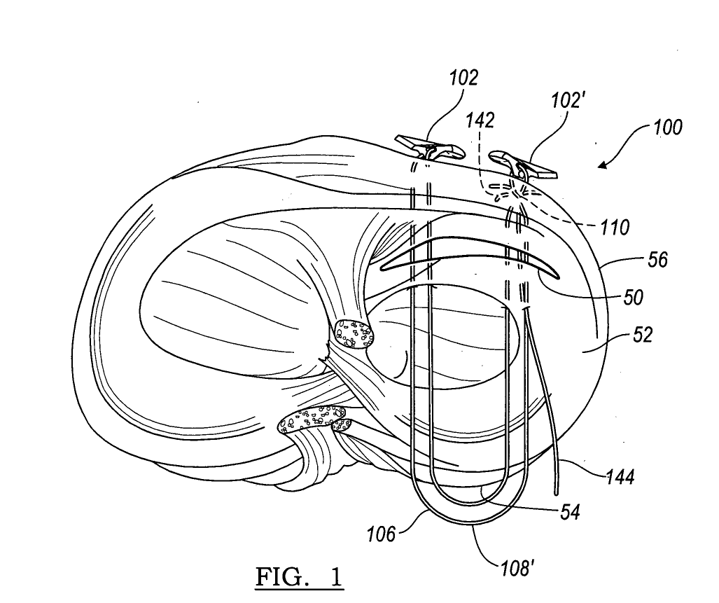

Referring to FIG. 1, an exemplary tissue repair assembly 100 according to the present teachings is illustrated for repairing a tear 50 in a tissue 52, such as a meniscus. The tissue repair assembly 100 may generally include two anchors 102, 102′ that are implanted at a distance apart from an anterior surface 54 of the tissue 52 to a posterior surface 56 of the tissue 52 across the tear 50. A flexible member 106, such as a suture, is pre-assembled between and connects the anchors 102, 102′. The flexible member 106 defines a main loop 108′ and is tied to itself by a self-locking slip knot 110 that terminates in one short free end 142 and one long free end 144. Pulling on the long free end 144 reduces the size of the main loop 108′, and brings the anchors 102,102′ closer to each other, thereby closing the tear 50, and locks the slip knot 110.

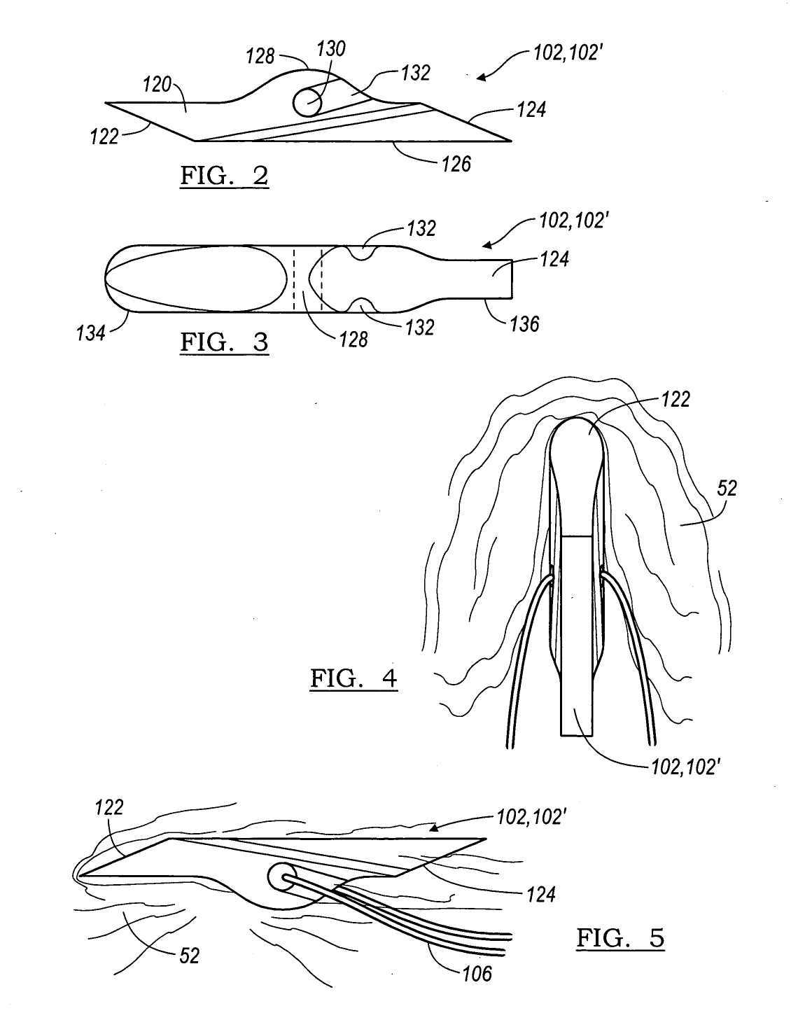

Each of the anchors 102, 102′ can be of the rotating (toggling) type, similar to the anchors described in the commonly owned U.S. Pat. No. 5,203,787, which is incorporated herein by reference. Referring to FIGS. 2-5, each anchor 102, 102′ can include a body 120 having a flat base 126 and first and second slanted end surfaces 122, 124. A curved projection 128 extends from the body 120 and defines a hole/eyelet 130 therethrough for receiving the flexible member 106. Guiding formations 132, such as grooves, extend from the hole 130 to guide the flexible member 106 at an angle away from the base 126. The first and second slanted end surfaces 122, 124 and the curved projection 128 help rotate the anchors 102, 102′ after implantation. The first end surface 122 is bounded by a rounded curved edge 134, and the second end surface 124 is bounded by a rectangular-shape edge 136. Each anchor 102, 102′ is implanted into tissue 52 with the curved edge 134 forward, as shown in FIG. 4, and then rotated about 90° from the insertion position of FIG. 4, to the implantation position shown in FIG. 5, with the curved projection 128 against the surface of the tissue 52. This anchor geometry facilitates insertion, improves grip and prevents backing out.

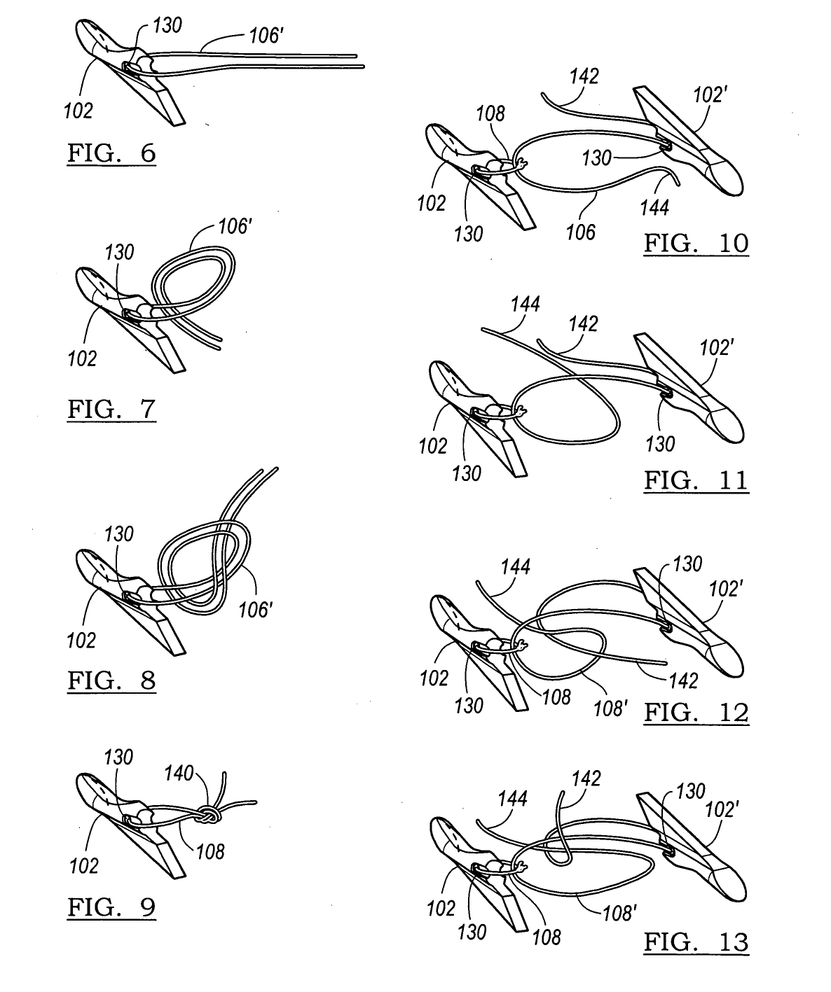

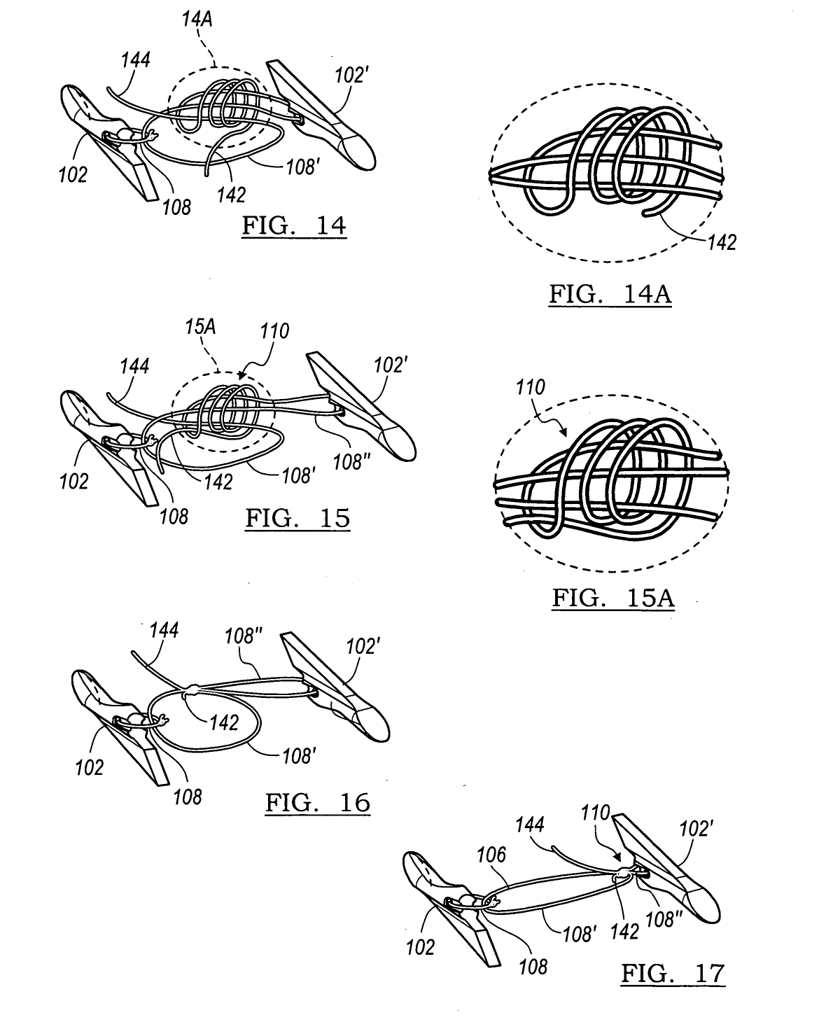

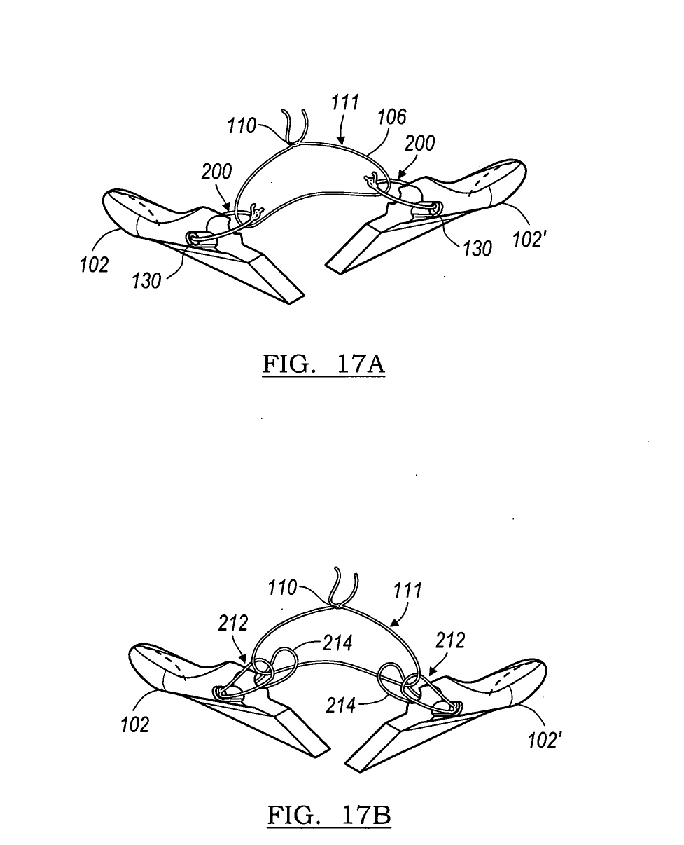

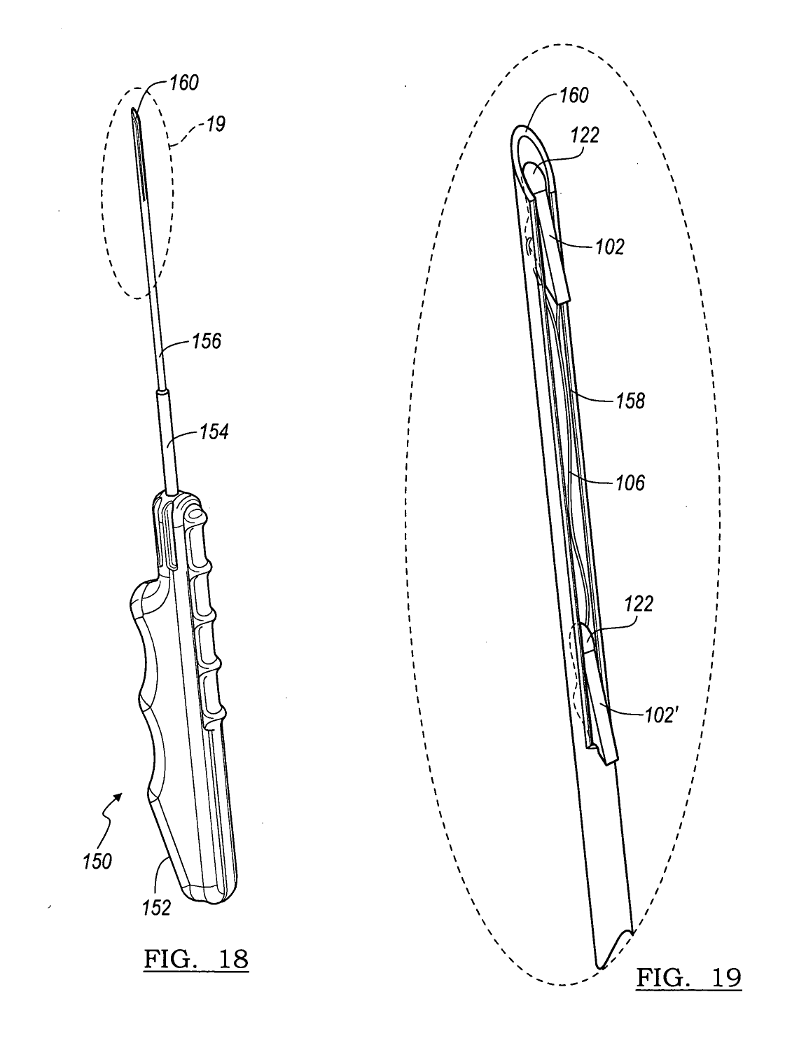

The flexible member 106 can be attached to the anchors 102, 102′ as illustrated in FIGS. 6-17. First, another piece of suture or other flexible member 106′ is passed through the hole 130 of the first anchor 102, and a first end loop 108 is formed using a conventional knot 140, as shown in FIGS. 6-9. Next, the flexible member 106 is used to connect the two anchors 102, 102′, as illustrated in FIGS. 10-17. The flexible member 106 is passed through the first end loop 108 of the first anchor 102 and through the hole 130 of the second anchor 102′, as shown in FIG. 10. FIGS. 11 and 12 illustrate forming the main loop 108′ with two free ends 142, 144. FIGS. 13, 14, 14A, 15, 15A and 16 show how the free end 142 is looped several times around a section of the flexible member 106 to form a slip knot 110 and a second end loop 108″ around the hole 130 of the second anchor 102. FIG. 17 shows the finished connection between the two anchors 102, 102′. The flexible member 106 passes through the first end loop 108 of the first anchor 102, and defines the main loop 108′ and the second end loop 108″ of the second anchor 102′. The self locking slip knot 110 is formed between the main loop 108′ and the second end loop 108″.

Referring to FIGS. 17A and 17B, another arrangement for connecting the flexible member 106 to the anchors 102, 102′ is illustrated, according to the present teachings. The self locking slip knot 110 is formed on a main loop 111 from a piece of flexible member 106, which is made to pass through two separate knotted end loops 200, as shown in FIG. 17A, or two separate continuous (unknotted) end loops 212, as shown in FIG. 17B. Each knotted end loop 200 comprises a piece of flexible member 106 that passes once through the anchor hole 130 and is knotted to itself at the ends. Each continuous end loop 212 passes through the anchor hole 130, such that it defines two sub-loops 214. The flexible member 106 of the main loop 111 is made to pass through each sub-loop 214 from each anchor 102, 102′.

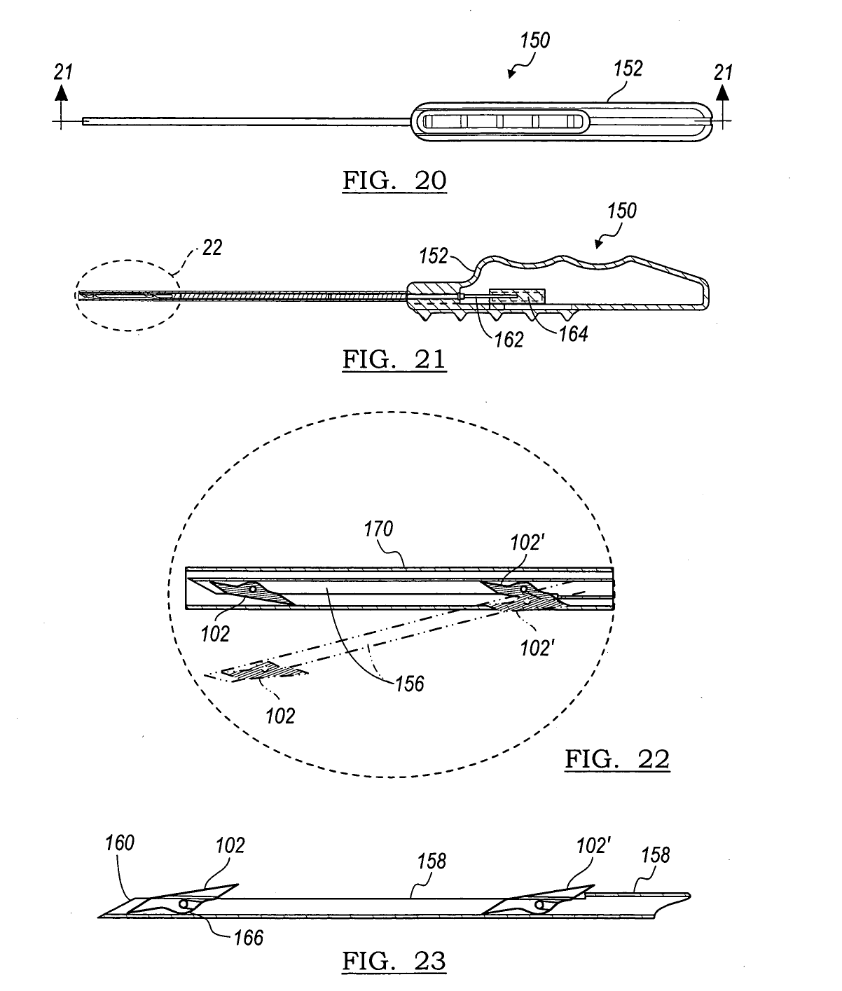

Referring to FIGS. 18-23, the repair assembly 100 can also include a delivery device or inserter 150 onto which the anchors 102, 102′, interconnected by the flexible member 106 as described above, can be loaded. The inserter 150 can include a handle 152 through which a tube 154 extends. Mounted on the tube 154 is a shaft 156 which can have a straight or curved portion, the latter illustrated in phantom lines in FIG. 22.

Referring to FIGS. 19, 22, and 23, the shaft 156 can have a longitudinal slot 158 ending at a slanted and curved open tip 160. The first and second anchors 102′, 102, with the flexible member 106 attached therebetween, can be inserted from the tip 160 into the slot 158 of the shaft, as shown in FIG. 19. The first anchor 102 is positioned adjacent the tip 160, such that it is the first to be implanted into the tissue 52. Referring to FIG. 21, a slider 162 in the inserter 150 can be operated with a thumb trigger 164 to push and to advance the second anchor 102 to the tip 160 for deployment after the first anchor 102 is deployed. To prevent accidental backing movement of the first anchor 102 along the slot 158 away from the tip 160 and toward the second anchor 102′, or accidental launching of the second anchor 102′ forward, a tab 166 can be formed by cutting along three sides the bottom surface of the shaft 156 near the tip 160 and bending the free end of the tab 166 slightly into the interior of the shaft 156. The force provided by the operation of the trigger 164 is sufficient to overcome the resistance provided by the tab 166 and launch the second anchor 102′.

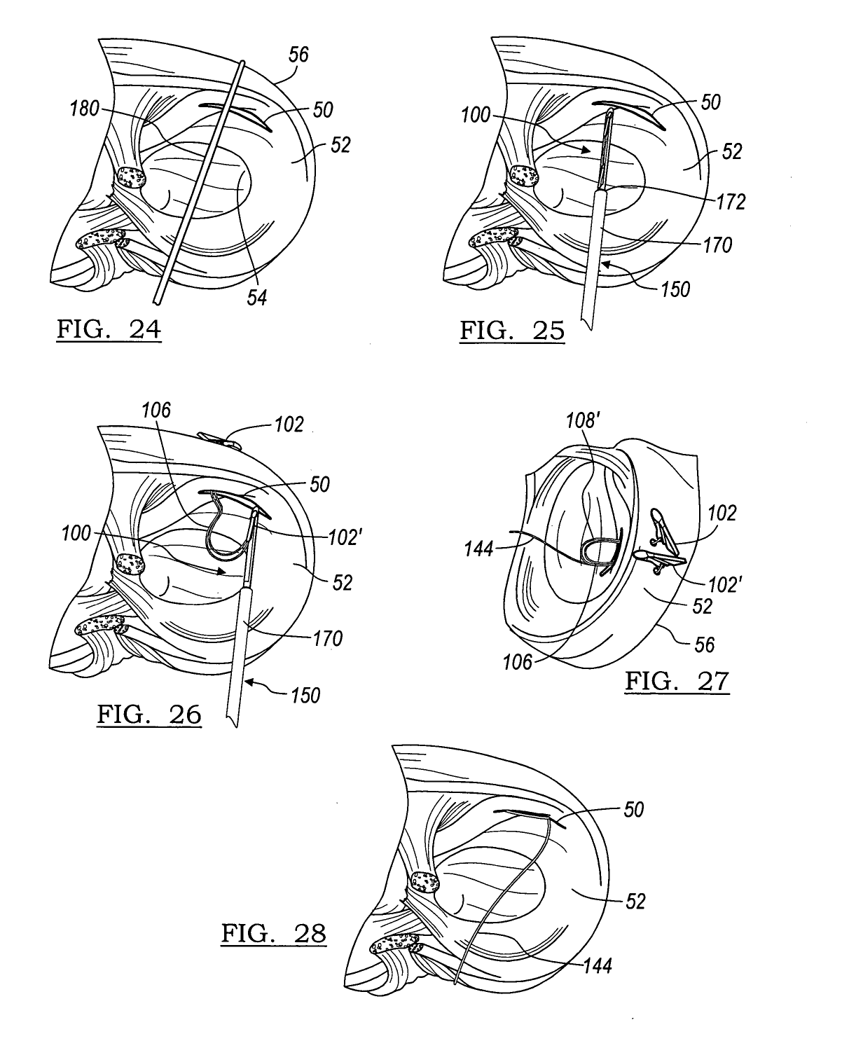

The shaft 156 can be provided with a tubular sleeve 170, shown in FIG. 22. The sleeve 170 can cover the shaft 156 from the tube 154 to the tip 160 and can be used as a protective cover before use, and/or as a depth delimiter by cutting the sleeve 170 to an appropriate depth after determining an insertion depth using a calibrated probe 180, as shown in FIG. 24.

Referring to FIGS. 24-28, the operation of the tissue repair assembly 100 is illustrated for, but not limited to, an exemplary application for meniscus repair. The insertion depth for the anchors 102, 102′ through the tear 50 in the tissue 52 can be determined using the calibrated probe 180, as shown in FIG. 24. The insertion depth can be measured from the anterior surface 54 to the posterior surface 56 of the meniscal tissue 52 across the tear 50. Referring to FIG. 25, after the insertion depth is determined, the depth delimiting sleeve 170 is cut to an appropriate length, and the inserter 150 is pushed through the tissue 52 and across the tear 50 until an end 172 of the sleeve 170 reaches the tissue 52. At this position, withdrawing the inserter from the tissue 52 causes the first anchor 102′ to be deployed and implanted against the posterior surface 56 of the tissue 52, as shown in FIG. 26.

After the first anchor 102 is implanted, the slider 162 is pushed with the trigger 164 to advance the second anchor 102′ past the tab 166 to the tip 160 of the shaft 156, as shown in FIG. 26. The inserter 150 is then pushed through the tissue 52 across the tear 50 at a distance away from the position of implantation of the first anchor 102. Withdrawing the inserter 150 deploys the second anchor 102′ and causes the second anchor 102′ to seat against the posterior surface 56 of the tissue as shown in FIG. 27. The slip knot 110 remains embedded in the tissue 52 under or onto the posterior surface 56. Referring to FIG. 28, tensioning the long free end 144 of the flexible member 106, tightens the main loop 108′, brings the anchors 102, 102′ closer together, and reduces the tear 50.

The rotatable anchors 102, 102′ and the threaded fastener 300 can be made from biocompatible materials, including titanium alloys or various resorbable materials, depending on the application. The shaft 156 can be made from biocompatible materials including metals such as stainless steel, such as high strength and toughness Custom 465® stainless alloy from Carpenter Technology Corp., Reading, Pa. The flexible member 106 can be made from appropriate biocompatible and/or resorbable materials including flat-braided, tubular polyester, or other suture materials known in the art.

It will be appreciated by those skilled in the art that the repair assembly 100 can be used to provide fast and convenient suturing and repair of tears in soft tissue, while keeping the anchoring devices, including the anchors 102, 102′ and the knot 110, away from the repair site and any articulating surfaces, thereby avoiding any possible functional interference, irritation, or and discomfort at the repair site.

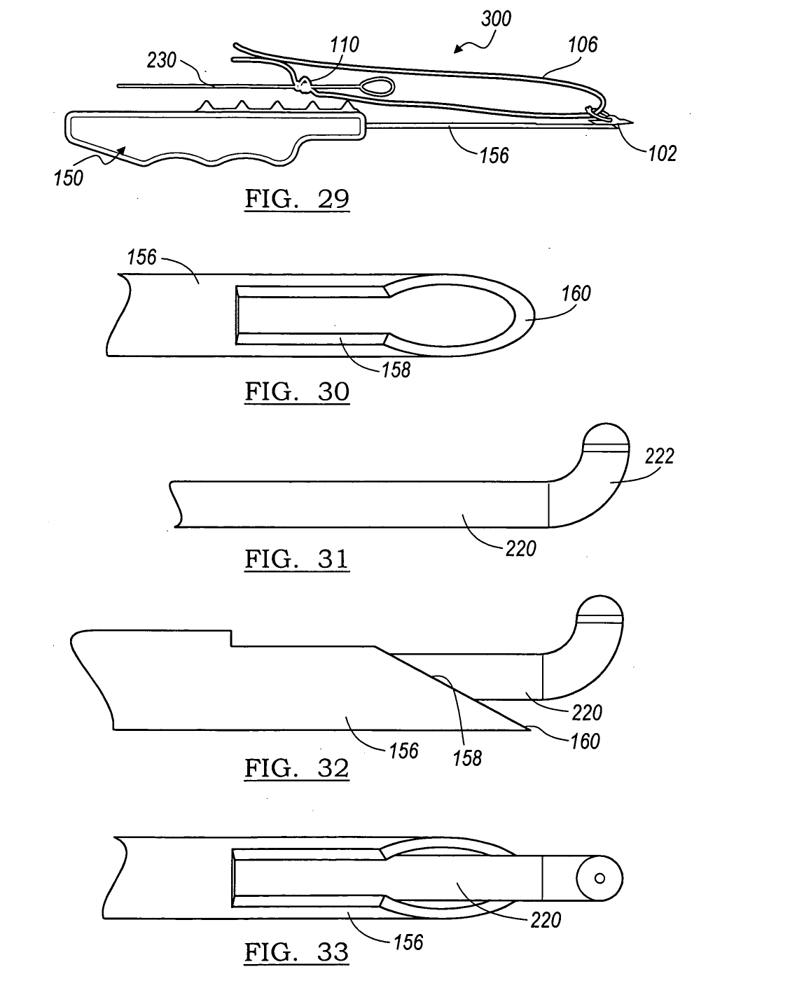

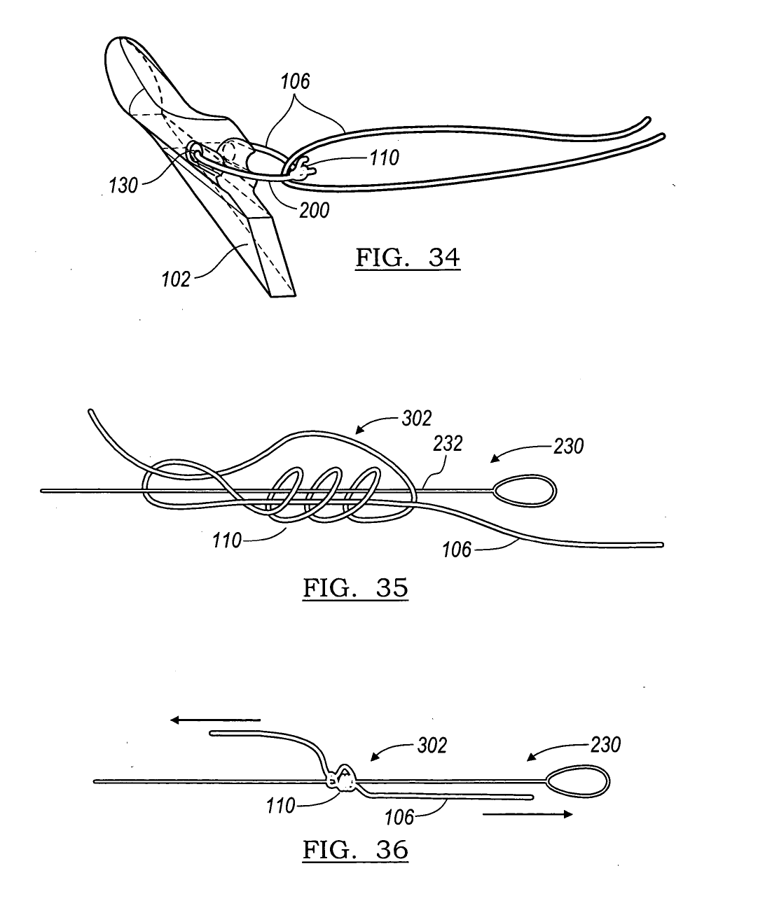

Referring to FIGS. 29-36, another tissue repair assembly 300 according to the present teachings can be used with a single anchor 102, two anchors 102, or more than two anchors 102 as desired by the surgeon. The repair assembly 300 can include the inserter 150 or similar inserter having shaft 156, longitudinal slot 158 and tip 160, as shown in FIG. 30, a capturing probe 220 illustrated in FIGS. 31-33, and a pre-tied knot assembly 302, illustrated in FIGS. 35 and 36. The capturing probe 220 can be moveably received in the shaft 156 and can be advanced out of the tip 160 of the shaft 156. The capturing probe 220 can include a hooked portion 222 and can be used to capture the flexible member 106.

Referring to FIGS. 35 and 36, the pre-tied knot assembly 302 can include a suture passer 230 having a body 232 and a slip knot 110 pre-tied on the body. The pre-tied knot assembly 302 and associated methods are disclosed and described in commonly assigned U.S. patent application Ser. No. 10/921,036, filed Aug. 18, 2004, which is incorporated herein by reference.

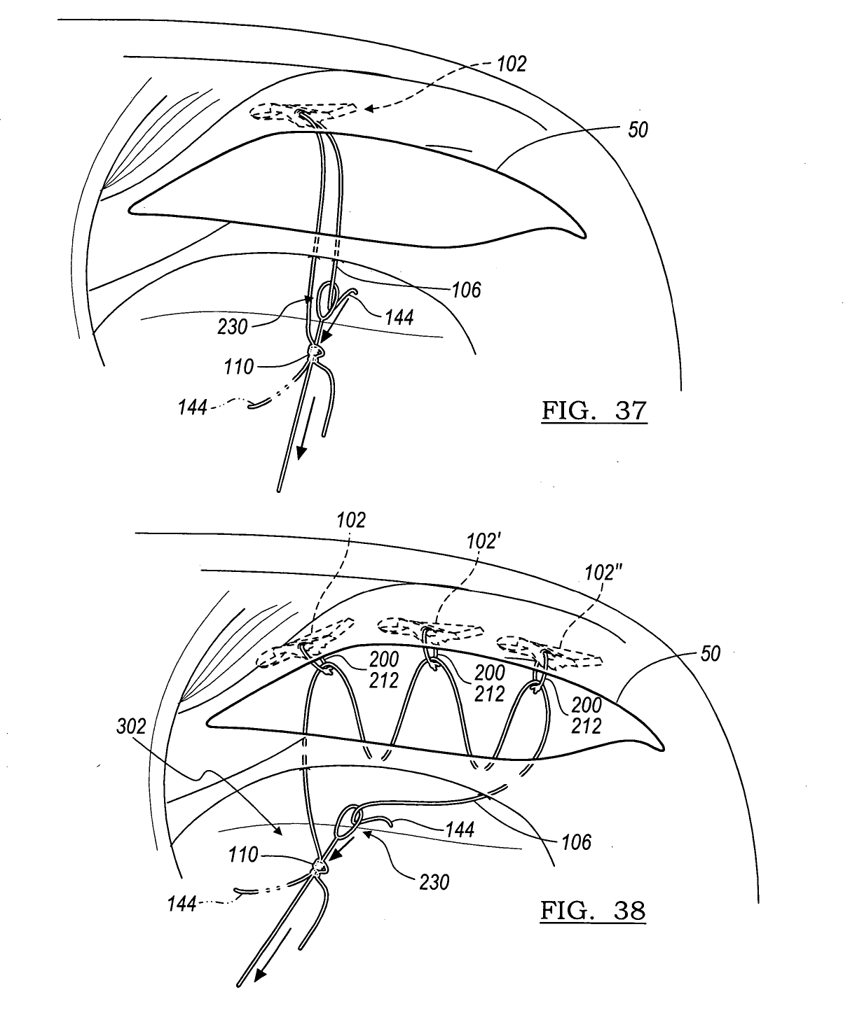

Referring to FIGS. 37 and 38, the tissue repair assembly 300 can be used to insert one anchor 102, or multiple anchors (three anchors 102, 102′, 102″ are illustrated in FIG. 38) similarly to the operation of the tissue repair assembly 100 described above in reference to FIGS. 24-28. Referring to FIG. 37 for use with a single anchor 102, the anchor 102 is inserted through tissue using the inserter 150 of the tissue repair assembly 100. The inserter 150 is then withdrawn, re-inserted in a secondary location and advanced to the open tear 50. Using visualization through the tear 50, the free end 144 of the flexible member 106 is captured by the capturing probe 220 and withdrawn through the secondary location. Using the suture passer 230, the free end of the flexible member 106 can be pulled through the slip knot 110 of the pre-tied knot assembly 302. The slip knot 110 is tightened, reducing the tear 50, and the remaining portion of the flexible member 106 is excised.

Referring to FIG. 38 for use with multiple anchors 102, 102′, 102″, and so on, as desired, the free end 144 of the flexible member 106 from the first anchor 102 is passed through the closed flexible loop 200 or 212, such as the loops 200 or 212 illustrated in FIGS. 17A and 17B, of the second anchor 102′, which is then inserted in a second location, and so on. When the last anchor has been inserted, the free end 144 is passed through the slip knot 110 using the suture passer 230 of the pre-tied knot assembly 302. The slip knot 110 is tightened, reducing the tear 50, and the remaining portion of the flexible member 106 is excised.

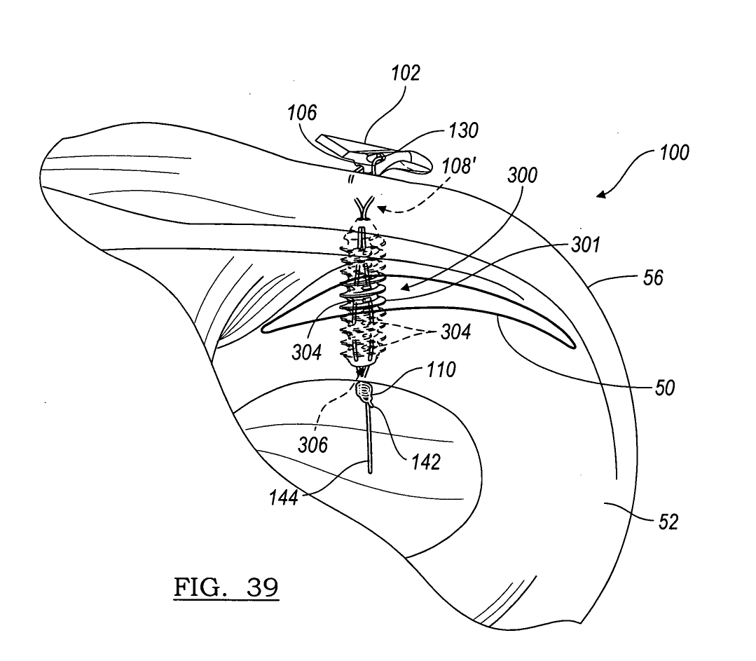

Referring to FIG. 39, in one aspect, the tissue repair assembly 100 can include a flexible member 106 connecting at least one rotatable/toggle anchor 102 to a cannulated fastener 300. The rotatable anchor 102 can be seated on the posterior surface 56 of the tissue 52, such as, for example a meniscus, for repairing a tear 50, which can be a vertical or bucket handle tear, as illustrated in FIG. 39. The fastener 300 can be a resorbable threaded fastener, such as, for example, the threaded fastener disclosed in pending co-owned patent application entitled Tissue Fixation Device, U.S. Ser. No. 11/294,694, and filed Dec. 5, 2005, the disclosure of which is incorporated by reference herein. The fastener 300 can include a helical or other thread 301 having blunt, rounded, or generally non-cutting edges, and a longitudinal bore 306 through which the ends 142, 144 of the flexible member 106 can be passed before forming a sliding self-locking knot, such as the knot 110 discussed above.

The fastener 300 can also include a plurality of perforations or other apertures 304 between the windings of the thread 301. The apertures 304 can provide flow communication between tissue outside the fastener 300 and the interior of the bore 306. The bore 306 of the fastener 300 can provide a path for blood flow along the fastener 300 between various regions of the tissue 52, such as, for example, between a highly vascular zone (red-red) and a less vascular zone (red-white, or white-white) of a meniscus. The apertures 304 can allow blood flow in and out of the fastener 300. Additionally, the bore 306 and the apertures 304 of the threaded fastener 300 can allow flow of other biological fluids either indigenous to the tissue or injectable through the fastener 300 for medicinal, therapeutic or other purposes. Injectable fluids can include, for example, pharmaceutical or biological agents and compositions, including agents with anti-bacterial or anti-microbial activities, pain relievers, healing or growth promoting agents, and other fluids or gels.

The flexible member 106 can pass directly through the hole 130 of the rotatable anchor 102, as illustrated in FIG. 39, such that both ends of the flexible member/suture 106 pass through the bore 306 of the fastener 300, defining a main suture loop 108′ terminating at the slip knot 110 or other retaining structure. When the flexible member 106 is tensioned, the tear 50 is reduced and the slip knot 110 is tightened against the fastener 300. The main suture loop 108′ can also be attached to the rotatable anchor 102 indirectly, by passing through a smaller fixed loop which is attached to the hole 130, similar to the end loop 108 shown in FIG. 12. it will be appreciated that other means for securing the flexible member 106 can also be used instead or in addition the slip knot 110, such as, for example, buttons, anchors, or other retaining structure.

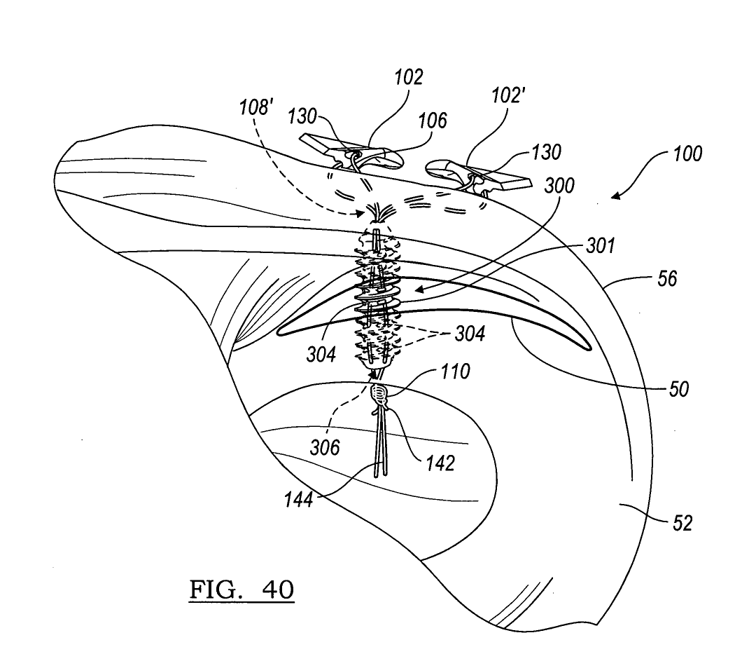

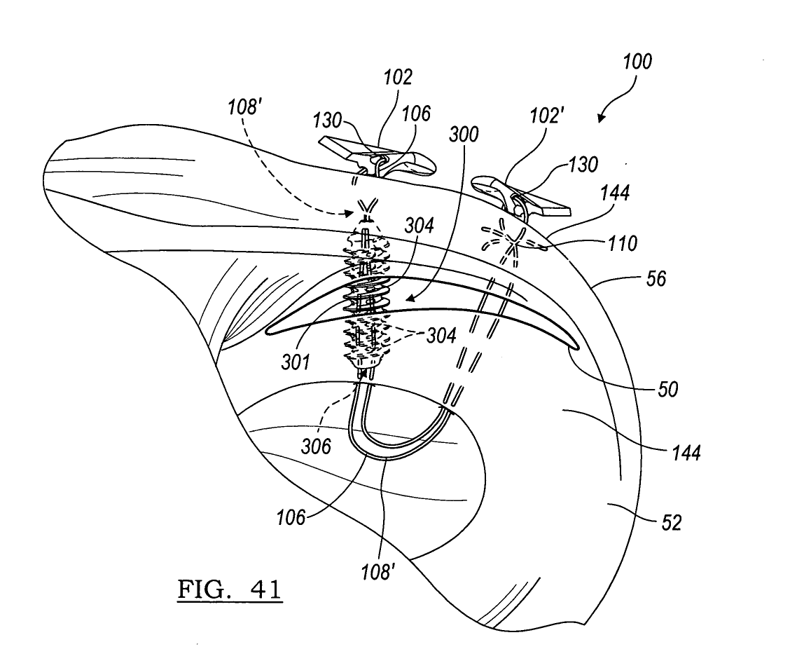

The rotatable anchor 102 can be inserted through the tear 50 and positioned on the posterior surface 56 of the tissue 52 using any of the instruments and methods discussed herein. More than one set of rotatable anchors 102 and corresponding threaded fasteners 300 can be used separately and independently, as desired by the surgeon, and in the manner illustrated in FIG. 39 for a single set of rotatable anchor 102 and threaded fastener 300, for repairing a particular tear. Several rotatable anchors 102 can also be used with a single fastener 300 to close the tear, with the flexible member 106 passing through each of the rotatable anchors 102, 102′ and the tear 50 before passing through the bore 306 of the fastener 300, as illustrated in FIG. 40 for two rotatable anchors 102, 102′. The threaded fastener 300 can also be used with two or more anchors 102, 102′, such that the suture loop 108′ passes through the first anchor 102, the tear 50 and the threaded fastener 300 before passing through the second rotatable anchor 102′, as illustrated in FIG. 41.

The threaded fastener 300 can also be preloaded on the inserter 150 and be connected to one or more rotatable anchors 102 through the flexible member 106, or can be threadably inserted separately. The threaded fastener 300 can be deployed from the inserter 150 through the tear 50 in a manner similar to the deployment of the rotatable anchors 102 described above, although the threaded fastener 300 does not exit the posterior surface 56 of the tissue 52 and does nor rotate, but remains lodged into the tissue 52 upon deployment, and after the inserter is withdrawn, as shown in FIG. 39.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A tissue repair assembly for repairing tissue comprising:
a rotatable anchor for insertion through a tissue tear on a surface of the tissue;
a threaded fastener for insertion into the tissue, the threaded fastener having a longitudinal bore and a plurality of apertures communicating with the bore; and
a flexible member coupled to the rotatable anchor and passing through the longitudinal bore such that tensioning the flexible member compresses the tear.

2. The assembly of claim 1, wherein the flexible member defines a main loop extending from the anchor through the bore of the threaded fastener and terminating at the slip knot outside the bore.

3. The assembly of claim 2, wherein the main loop passes through an end loop coupled to the rotatable anchor.

4. The assembly of claim 1, wherein the rotatable anchor includes:
a substantially flat base;
a rounded slanted end;
a rectangular slanted end; and
a curved projection having a hole and guiding formations extending from the hole for guiding the flexible member.

5. The assembly of claim 4, wherein the rounded end, the slanted end and the curved projection are operable for rotating the rotatable anchor after or during implantation.

6. The assembly of claim 1, further comprising an inserter for implanting the anchor.

7. The assembly of claim 6, wherein the inserter includes a shaft having a longitudinal slot for receiving the anchor.

8. The assembly of claim 6, further including a depth delimiting sleeve.

9. The assembly of claim 6, further including a thumb-actuated slider for advancing another anchor.

10. The tissue repair assembly of claim 3, wherein the end loop comprises two sub-loops.

11. The tissue repair assembly of claim 1, wherein the threaded fastener is resorbable.

12. The tissue repair assembly of claim 1, further comprising a biologic fluid agent injectable through the longitudinal bore of the threaded fastener.

13. A method for repairing a tear in tissue, the method comprising:
providing an inserter having a shaft loaded with a rotatable anchor and a threaded fastener, the rotatable anchor and the threaded fastener connected with a flexible member passing through a bore of the fastener, the flexible member defining a self-locking slip knot outside the fastener and having a free end;
deploying the rotatable anchor through a first surface of the tissue across the tear onto a second surface of the tissue;
deploying the threaded fastener through the first surface of the tissue across the tear into the tissue; and
tensioning the free end of the flexible member to reduce the tear.

14. The method of claim 13, wherein deploying the rotatable anchor comprises:
inserting the inserter through the first surface of the tissue across the tear to the second surface of the tissue; and
withdrawing the inserter.

15. The method of claim 14, wherein withdrawing the inserter comprises rotating the rotatable anchor.

16. The method of claim 14, wherein deploying the threaded fastener comprises:
advancing the threaded fastener in a deployment position in the inserter;
inserting the inserter through the first surface of the tissue across the tear into the tissue;
threadably implanting the threaded fastener; and
withdrawing the inserter.

17. The method of claim 13, wherein tensioning the free end of the flexible member includes reducing the distance between the rotatable anchor and the threaded fastener.

18. The method of claim 13, wherein tensioning the free end of the flexible member includes reducing a length of a main loop of the flexible member between the rotatable anchor and the threaded fastener.

19. The method of claim 13, wherein the tissue is a meniscus, the first surface is an anterior surface of the meniscus, and the second surface is a posterior surface of the meniscus.

20. A method for repairing a tear in tissue, the method comprising:
providing a plurality of rotatable anchors and a threaded fastener connected with a flexible member, the flexible member passing through a bore of the threaded fastener and defining loop terminating at a self-locking slip knot;
inserting each rotatable anchor through a first surface of the tear onto a second surface of the tear such that the flexible member passes through the tear between successive anchors;
inserting the threaded fastener through the first surface of the tear into the tissue; and
tensioning the free end of the flexible member to reduce the tear and lock the slip-knot against the fastener.

21. The method of claim 20, further comprising allowing biological fluid flow through the bore of the threaded fastener and apertures thereon.

22. The method of claim 20, further comprising passing a free end of the flexible member through the slip knot using the suture passer.

23. The method of claim 20, wherein inserting comprises inserting using a pre-loaded inserter.
